(19) United States
(12) Patent Application Publication
(10) Pub. No.: US 2016/0375049 A1
(43) Pub. Date: Dec. 29, 2016

(54) COMPOSITIONS

(71) Applicant: GLOBALACORN LTD, London (GB)

(72) Inventor: Andrew David Miller, London (GB)

(21) Appl. No.: 15/039,714

(22) PCT Filed: Nov. 27, 2014
(86) PCT No.: PCT/GB2014/053521

§ 371 Date: May 26, 2016

(30) Foreign Application Priority Data
Nov. 27, 2013 (GB) 1320962.2

(51) Int. Cl. A61K 31/7084; A61K 9/00; A61K 9/70; A61K 31/167; A61K 9/127; A61K 31/09; C12N 15/113; A61K 31/245

(52) U.S. Cl. CPC A61K 31/7084; C12N 15/1138; A61K 9/0014; A61K 31/245; A61K 31/167; A61K 9/127; A61K 9/0021; A61K 31/09; A61K 9/7061; C12N 2320/51; C12N 2320/31; C12N 2310/14; C12N 2320/32

(57) ABSTRACT

The present invention relates to administration of a dinucleoside polyphosphate analogue or a pharmaceutically acceptable salt thereof, topically in a formulation comprising a suitable excipient or using a device for transdermal delivery, and/or combined with a nanoparticle carrier. The present invention also relates to the therapeutic use of such compositions or devices, in particular in the treatment of pain or epilepsy. The analogue may be combined with an anaesthetic (such as a salt form) or delivered in a nanoparticle.

### FIELD OF THE INVENTION

The present invention relates to administration of a dinucleoside polyphosphate analogue, or a pharmaceutically acceptable salt thereof, topically or transdermally in a formulation (comprising a suitable excipient) or capable of slow and/or sustained release, using a device for transdermal delivery, and/or combined with a nanoparticle carrier and/or anaesthetic. The present invention also relates to the therapeutic use such compositions or devices, in particular in the treatment of pain.

### BACKGROUND TO THE INVENTION

More than 270 million people worldwide suffer from chronic pain, which is still treated predominantly by opioids and non-steroidal anti-inflammatory drugs (NSAIDs). While there have been small improvements in both these areas, they still suffer from significant adverse side effects and dependency issues.

It is suggested that P2X3 receptors are involved in various states of chronic pain, including inflammatory and cancer-associated pain. Previous studies have shown that P2X3 antagonists or genetic deletion can have analgesic effects on inflammatory and neuropathic pain models. Several non-nucleotide antagonists may inhibit the activities of P2X3 receptors such as AF-353, a bacterial DHFR inhibitor, that is also a potent and selective non-competitive antagonist of P2X3 (Geyer et al, 2010). It has been shown to allosterically modulate the interaction of nucleic acids with P2X3 without being a competitive antagonist of α,β-meATP. A-317491 is a competitive antagonist of P2X3 and P2X 2/3, and binds to P2X3 receptors within a micromolar range of concentration (Jarvis et al, 2002). Systemic administration of A-317491 effectively reduced nociception in inflammatory and neuropathic pain models (Jarvis et al., 2002; McGaraughty et al., 2003). A-317491 also effectively blocked persistent pain in the formalin and acetic acid-induced abdominal constriction tests but was generally inactive in models of acute noxious stimulation. A-317491 is more efficient when injected intrathecally than in peripheral nervous system (Jarvis et al, 2002), indicating action within the central nervous system. RO-3, a non-competitive antagonist of P2X3 receptors, has been found to induce anti-nociception in animal models (Geyer et al., 2006). Purotoxin-1, a spider venom peptidic toxin, binds to P2X3 and exerts a selective inhibitory action on P2X3 receptors (Grishin et al., 2010), its binding mechanism is not well known.

However research into potent P2X3-selective ligands with reasonable bioavailability is still lacking. To date, no selective P2X3 receptor antagonists have been evaluated successfully in clinic for the relief of chronic nociceptive or neuropathic pain.

### SUMMARY OF THE INVENTION

The present invention relates to compositions, devices and methods which can enhance delivery and optimize bioavailabilty of dinucleoside polyphosphase analogues to a target.

Thus, in one aspect the present invention provides a pharmaceutical composition (that is adapted) for topical administration, or slow or sustained release, comprising a dinucleoside polyphosphate analogue, or a pharmaceutically acceptable salt thereof, and a pharmaceutically acceptable excipient. The composition may suitably be in the form of a solution, cream, foam, gel, lotion or ointment.

The present invention also provides a compound which is (a salt of) a dinucleoside polyphosphate analogue and or combined with an anaesthetic (compound). The compound may thus be combined with or comprise a suitable counter ion.

The present invention further provides a device for transdermal (or topical) delivery, comprising a dinucleoside polyphosphate analogue or a pharmaceutically acceptable salt thereof.

In one aspect, the present invention provides a composition, compound or a device for transdermal delivery as described above for use in treatment of the human or animal body by administration to the skin or an epithelial cell surface of a human or animal subject, such as administration in the form of a solution, cream, foam, gel, lotion or ointment, or by a device for transdermal delivery. In particular, the composition, compound or device are for use in the treatment of pain, as an anticonvulsant and/or as a seizure suppressant.

In another aspect, the present invention provides a pharmaceutical composition comprising a dinucleoside polyphosphate analogue or a pharmaceutically acceptable salt thereof, and/or combined with a nanoparticle carrier, and a pharmaceutically acceptable excipient. The present invention also provides a such a composition for use in treatment of the human or animal body, in particular for treatment of pain, as an anticonvulsant and/or as a seizure suppressant.

### DETAILED DESCRIPTION OF THE INVENTION

The invention uses dinucleoside polyphosphates, a family of compounds comprising two nucleoside moieties linked by a polyphosphate bridge. They can be represented by NpₙN, wherein N represents a nucleoside moiety, p represents a phosphate group and n is the number of phosphate groups (e.g. 2 to 7) Analogues of dinucleoside polyphosphates are compounds (typically synthetic) having a structure based on that of a dinucleoside polyphosphate, wherein one or more parts of the structure have been altered. For example the nucleobase, the sugar and/or the phosphate backbone may be modified, or partially or fully replaced, by another suitable moiety.

For example, one or more polyphosphate chain oxo-bridges may be replaced by a different bridge to increase the biological half-life of the compound in vivo. Such analogues may be designed to provide stability and/or biocompatibility. To achieve this, the analogue should be resistant to decomposition by biological systems in vivo. For example, the analogue may have increased hydrolytic stability, i.e. resistance to the breakdown of the molecule by specific enzyme cleavage (e.g. by one or more types of nucleotidase) and/or non-specific hydrolysis.

Preferably the compounds (or their salts) are diadenosine polyphosphates (e.g. of the type ApₙAs; where n is 2-7), such as naturally occurring purinergic ligands consisting of two adenosine moieties bridged by a chain of two or more phosphate residues attached at the 5′-position of each ribose ring. In particular, P¹,P⁴-diadenosine tetraphosphate (Ap₄A) and P¹,P⁵-diadenosine pentaphosphate (Ap₅A) are contemplated. These are present in high concentrations endogenously in the secretory granules of chromaffin cells and in rat brain synaptic terminals. Upon depolarization, ApₙAs are released in a Ca²⁺-dependent manner and their potential role as neurotransmitters has been proposed. However, in spite of being well known for many years, pure functions of ApₙAs have been difficult to define because of both specific enzymatic cleavage and nonspecific hydrolytic breakdown. ApₙA analogues can be more stable than naturally occurring diadenosine polyphosphates with respect to both specific enzymatic and nonspecific hydrolytic breakdown.

### Preferred Compounds

Preferably, the dinucleoside polyphosphate (of the NPₙN type) for use in the present invention (which includes salts thereof) is a compound of formula (I):

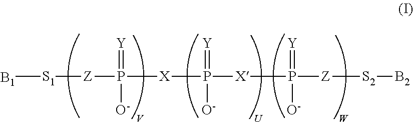

or a pharmaceutically acceptable salt thereof,
wherein X, X′ and Z are independently selected from

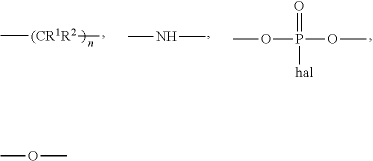

wherein R¹ and R² are independently selected from hydrogen, halogen, hydroxyl, cyano or an unsubstituted group selected from C₁₋₃ haloalkyl, C₁₋₃ alkyl, C₁₋₄ aminoalkyl and C₁₋₄ hydroxyalkyl, and n is selected from 1, 2, 3, 4, 5 and 6;
each Y is independently selected from ═S and ═O;
B₁ and B₂ are independently selected from a 5- to 7-membered carbon-nitrogen heteroaryl group which may be unfused or fused to a further 5- to 7-membered carbon-nitrogen heteroaryl group
S₁ and S₂ are independently selected from a bond, C₁₋₆ alkylene, C₂₋₆ alkenylene, C₂₋₆ alkynylene and a moiety of formula (II):

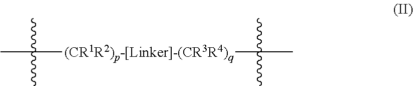

wherein
R¹, R², R³ and R⁴ independently represent hydrogen, halogen, hydroxyl, cyano or an unsubstituted group selected from C₁₋₃ haloalkyl, C₁₋₃ alkyl, C₁₋₄ aminoalkyl and C₁₋₄ hydroxyalkyl;
p and q independently represent 0, 1, 2 or 3, preferably 0, 1 or 2; and
[Linker] represents:
   (i) —O—, —S—, —C═O— or —NH—;
   (ii) C₁₋₄ alkylene, C₂₋₄ alkenylene or C₂₋₄ alkynylene, which may optionally contain or terminate in an ether (—O—), thioether (—S—), carbonyl (—C═O—) or amino (—NH—) link, and which are optionally substituted with one or more groups selected from hydrogen, hydroxyl, halogen, cyano, —NR⁵R⁶ or an unsubstituted group selected from C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ aminoalkyl, C₁₋₄ hydroxyalkyl, C₁₋₄ acyl and C₁₋₄ alkyl-NR⁵R⁶ groups, wherein R⁵ and R⁶ are the same or different and represent hydrogen or unsubstituted C₁₋₂ alkyl; or
   (iii) a 5 to 7 membered heterocyclyl, carbocyclyl or aryl group, which may be optionally substituted with one or more groups selected from hydrogen, hydroxyl, halogen, cyano, —NR⁵R⁶ or an unsubstituted group selected from C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ aminoalkyl, C₁₋₄ hydroxyalkyl, C₁₋₄ acyl and C₁₋₄ alkyl-NR⁵R⁶ groups, wherein R⁵ and R⁶ are the same or different and represent hydrogen or unsubstituted C₁₋₂ alkyl;
      V is selected from 0, 1, 2, 3, 4 and 5;
      U is selected from 0, 1, 2, 3, 4 and 5;
      W is selected from 0, 1, 2, 3, 4 and 5; and
      V plus U plus W is an integer from 2 to 7.

As used herein, a C₁₋₄ alkyl group or moiety is a linear or branched alkyl group or moiety containing from 1 to 4 carbon atoms. Examples of C₁₋₄ alkyl groups include methyl, ethyl, n-propyl, i-propyl, n-butyl, i-butyl and t-butyl.

As used herein, a C₂₋₄ alkenyl group or moiety is a linear or branched alkenyl group or moiety having at least one double bond of either E or Z stereochemistry where applicable and containing from 2 to 4 carbon atoms, such as —CH═CH₂ or —CH₂—CH═CH₂, —CH₂—CH₂—CH═CH₂, —CH₂—CH═CH—CH₃, —CH═C(CH₃)—CH₃ and —CH₂—C(CH₃)═CH₂.

As used herein, a C₁₋₆ alkylene group or moiety is a linear or branched alkylene group or moiety, for example a C₁₋₄ alkylene group or moiety. Examples include methylene, n-ethylene, n-propylene and —C(CH₃)₂— groups and moieties.

As used herein, a C₂₋₆ alkenylene group or moiety is a linear or branched alkenylene group or moiety, for example a C₂₋₄ alkenylene group or moiety. Examples include —CH═CH—, —CH═CH—CH₂—, —CH₂—CH═CH— and —CH═CH—CH═CH—.

As used herein, a C₂₋₆ alkynylene group or moiety is a linear or branched alkynylene group or moiety, for example a C₂₋₄ alkynylene group or moiety. Examples include —C≡C—, —C≡C—CH₂— and —CH₂—C≡C—.

As used herein, a halogen atom is chlorine, fluorine, bromine or iodine.

As used herein, a C₁₋₄ alkoxy group or C₂₋₄ alkenyloxy group is typically a said C₁₋₄ alkyl group or a said C₂₋₄ alkenyl group respectively which is attached to an oxygen atom.

A haloalkyl or haloalkenyl group is typically a said alkyl or alkenyl group respectively which is substituted by one or more said halogen atoms. Typically, it is substituted by 1, 2 or 3 said halogen atoms. Preferred haloalkyl groups include perhaloalkyl groups such as —CX₃ wherein X is a said halogen atom, for example chlorine or fluorine.

Preferably, a C₁₋₄ or C₁₋₃ haloalkyl group as used herein is a C₁₋₃ fluoroalkyl or C₁₋₃ chloroalkyl group, more preferably a C₁₋₃ fluoroalkyl group.

As used herein, a C₁₋₄ aminoalkyl group is a C₁₋₄ alkyl group substituted by one or more amino groups. Typically, it is substituted by one, two or three amino groups. Preferably, it is substituted by a single amino group.

As used herein, a C₁₋₄ hydroxyalkyl group is a C₁₋₄ alkyl group substituted by one or more hydroxy groups. Typically, it is substituted by one, two or three hydroxy groups. Preferably, it is substituted by a single hydroxy group.

As used herein, a C₁₋₄ acyl group is a group —C(═O)R, wherein R is a said C₁₋₄ alkyl group.

As used herein, a 5 to 7 membered heterocyclyl group includes heteroaryl groups, and in its non-aromatic meaning relates to a saturated or unsaturated non-aromatic moiety having 5, 6 or 7 ring atoms and containing one or more, for example 1 or 2, heteroatoms selected from S, N and O, preferably O. Illustrative of such moieties are tetrahydrofuranyl and tetrahydropyranyl. For example, the heterocyclic ring may be a furanose or pyranose ring.

As used herein, a 5- to 7-membered carbon-nitrogen heteroaryl group is a monocyclic 5- to 7-membered aromatic ring, such as a 5- or 6-membered ring, containing at least one nitrogen atom, for example 1, 2, 3 or 4 nitrogen atoms. The 5- to 7-membered carbon-nitrogen heteroaryl group may be fused to another 5- to 7-membered carbon-nitrogen heteroaryl group.

As used herein, a 5 to 7 membered carbocyclyl group is a non-aromatic, saturated or unsaturated hydrocarbon ring having from 5 to 7 carbon atoms. Preferably it is a saturated or mono-unsaturated hydrocarbon ring (i.e. a cycloalkyl moiety or a cycloalkenyl moiety) having from 5 to 7 carbon atoms. Examples include cyclopentyl, cyclohexyl, cyclopentenyl and cyclohexenyl.

As used herein, a 5 to 7 membered aryl group is a monocyclic, 5- to 7-membered aromatic hydrocarbon ring having from 5 to 7 carbon atoms, for example phenyl.

In one aspect X and X′ are independently —NH—.

In one aspect X and X′ are independently

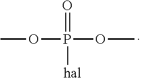

In one aspect X and X′ are independently

—(CR¹R²)ₙ—,

wherein at least one of R¹ and R² is H, Cl, Br or F.

Preferably both R¹ and R² are H.

Preferably n is 1, 2 or 3, preferably 1 or 2.

Preferably at least one of X and X′ is not —O—, i.e. not all X and X′ are —O—.

Preferably X and X′ are independently selected from NH and

—(CR¹R²)ₙ—

wherein R¹ and R² are both H and n is 1 or 2.

In one aspect at least one Y is ═S.

In one aspect each Y group is ═S.

In one aspect at least one Y is ═O.

Preferably each Y group is ═O.

In one aspect at least one Z is

—(CR¹R²)ₙ—.

In one aspect each Z is

—(CR¹R²)ₙ—

wherein at least one of R¹ and R² is H, Cl, Br or F.

Preferably both R¹ and R² are H. Thus, in one aspect Z is

—(CR¹R²)ₙ—

and R¹ and R² are both H.

Preferably n is 1, 2 or 3, preferably 1 or 2.

In one aspect at least one Z is —NH—.

In one aspect each Z is —NH—.

In one aspect at least one Z is —O—.

Preferably each Z is —O—.

B₁ and B₂ are preferably independently selected from purine and pyrimidine nucleic acid bases, preferably adenine, guanine, thymine, cytosine, uracil, hypoxanthine, xanthine, 1-methyladenine, 7-methylguanine, 2-N,N-dimethylguanine, 5-methylcytosine or 5,6-dihydrouracil. Uracil may be attached to S₁ or S₂ via N (i.e. uridine structure) or C (i.e. pseudouridine structure).

Preferably, B₁ and B₂ are independently selected from adenine, guanine, and uracil.

Preferably at least one of B₁ and B₂ is adenine.

Thus, for example, at least one of B₁ and B₂ may be adenine and the other of B₁ and B₂ may be guanine, or at least one of B₁ and B₂ may be adenine and the other of B₁ and B₂ may be uracil.

Preferably, B₁ and B₂ are both adenine, or one of B₁ and B₂ is adenine and the other is guanine.

S₁ and S₂ are preferably independently selected from a bond, C₁₋₆ alkylene, C₂₋₆ alkenylene, C₂₋₆ alkynylene and a moiety of formula (III) or (IV):

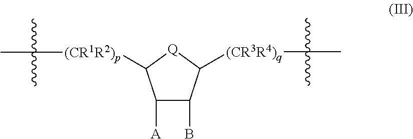

wherein
R¹, R², R³ and R⁴ independently represent hydrogen, halogen, hydroxyl, cyano or an unsubstituted group selected from C₁₋₃ haloalkyl, C₁₋₃ alkyl, C₁₋₄ aminoalkyl and C₁₋₄ hydroxyalkyl;
p and q independently represent 0 or 1;
Q represents —O—, —S—, —C═O—, —NH— or CH₂; and
A and B independently represent hydrogen, hydroxyl, halogen, or an unsubstituted group selected from C₁₋₄ alkoxy, C₁₋₄ aminoalkyl, C₁₋₄ hydroxyalkyl, C₁₋₄ acyl and —NR⁵R⁶ groups, wherein R⁵ and R⁶ are the same or different and represent hydrogen or unsubstituted C₁₋₂ alkyl;

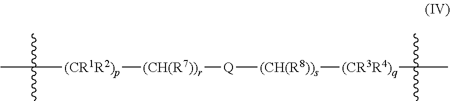

wherein
R¹, R², R³ and R⁴ independently represent hydrogen, halogen, cyano or an unsubstituted group selected from C₁₋₃ haloalkyl, C₁₋₃ alkyl, C₁₋₄ aminoalkyl and C₁₋₄ hydroxyalkyl;
Q represents —O—, —S—, —C═O—, —NH— or CH₂; and
R⁷ and R⁸ independently represent hydrogen, hydroxyl, halogen, cyano, —NR⁵R⁶ or an unsubstituted group selected from C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ aminoalkyl, C₁₋₄ hydroxyalkyl, C₁₋₄ acyl and C₁₋₄ alkyl-NR⁵R⁶ groups, wherein R⁵ and R⁶ are the same or different and represent hydrogen or unsubstituted C₁₋₂ alkyl; and
p, q, r and s independently represent 0 or 1.

S₁ and S₂ are preferably independently selected from a moiety of formula (III) or (IV) as set out above, in which preferably:
R¹, R², R³ and R⁴ independently represent hydrogen, fluoro, chloro, or unsubstituted C₁₋₃ alkyl; more preferably hydrogen;
Q represents —O—;
A and B independently represent hydrogen, hydroxyl, fluoro, chloro, methoxy, formyl or NH₂, more preferably hydrogen or hydroxyl; and
R⁷ and R⁸ independently represent hydrogen, hydroxyl, fluoro, chloro, or an unsubstituted group selected from C₁₋₄ alkyl, C₁₋₄ haloalkyl, C₁₋₄ hydroxyalkyl and C₁₋₄ alkyl-NH₂, more preferably hydrogen, hydroxyl or unsubstituted methyl, ethyl, —CH₂OH or —CH₂CH₂OH.

S₁ and S₂ may preferably be independently selected from D-ribofuranose, 2′-deoxy-D-ribofuranose, 3′-deoxy-D-ribofuranose, L-arabinofuranose (corresponding to moieties of formula (III)), and ring opened forms thereof (corresponding to moieties of formula (IV)).

In one preferred embodiment, at least one of S₁ and S₂ is D-ribofuranose, i.e. a moiety of formula (III′) in which R¹ and R² are hydrogen, p is 1, q is 0, Q is —O— and A and B are hydroxyl:

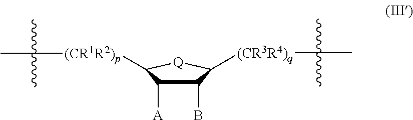

When S₁ and/or S₂ is a ring opened form, the ring opening is preferably between the 2′ and 3′ positions of the D-ribofuranose, 2′-deoxy-D-ribofuranose, 3′-deoxy-D-ribofuranose or L-arabinofuranose ring.

In one preferred embodiment, at least one of S₁ and S₂ is a ring opened form of D-ribofuranose, for example a moiety of formula (IV) in which R¹ and R² are hydrogen, p is 1, q is 0, Q is —O—, r is 1, s is 1 and R⁷ and R⁸ are each —CH₂OH.

Preferably S₁ and S₂ are the same. Thus preferably, S₁ and S₂ are both D-ribofuranose or both a ring opened form of D-ribofuranose as described above.

The sum of V, U and W may be 2, 3, 4, 5, 6 or 7.

Preferably V plus U plus W is 4 or 5.

Preferably U is 0, 1 or 2.

Preferably V is 2.

Preferably W is 2.

In a preferred embodiment, U is 0. Thus the dinucleoside polyphosphate for use in the present invention is preferably a compound of formula (I′):

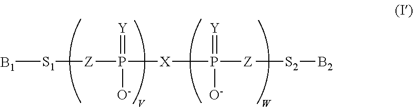

wherein all symbols are as defined above, X is not —O— and V plus W is a integer from 2 to 7.

Thus, the sum of V and W in formula (I′) may be 2, 3, 4, 5, 6 or 7. Preferably V plus W is 4 or 5. Preferably V is 2 and/or W is 2.

In a preferred embodiment, each Y is ═O and each Z is —O—.

In a more preferred embodiment, each Y is ═O and each Z is —O—, and both S₁ and S₂ are a moiety of formula (III) or (IV) as set out above. Preferably, both S₁ and S₂ are the same and are both D-ribofuranose or both a ring opened form of D-ribofuranose. Thus the dinucleoside polyphosphate analogue of the present invention is preferably a compound of formula (IA) or (IB):

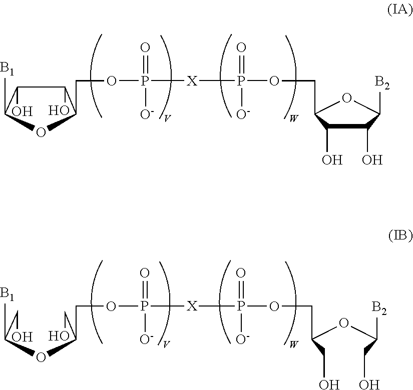

Preferably, the dinucleoside polyphosphate analogue of the present invention is a compound of formula (IA) or (IB) wherein V plus W is 4 or 5. More preferably, the dinucleoside polyphosphate analogue of the present invention is a compound of formula (IA) or (IB) wherein at least one of B₁ and B₂ is adenine, or one of B₁ and B₂ is adenine and the other is guanine.

Thus, in a more preferred embodiment, each Y is ═O and each Z is —O—, both S₁ and S₂ are the same and are both D-ribofuranose or both a ring opened form of D-ribofuranose, and B₁ and B₂ are both adenine, or one of B₁ and B₂ is adenine and the other is guanine. Thus the dinucleoside polyphosphate analogue of the present invention is preferably a dinucleoside polyphosphate compound of formula (IC) to (IF):

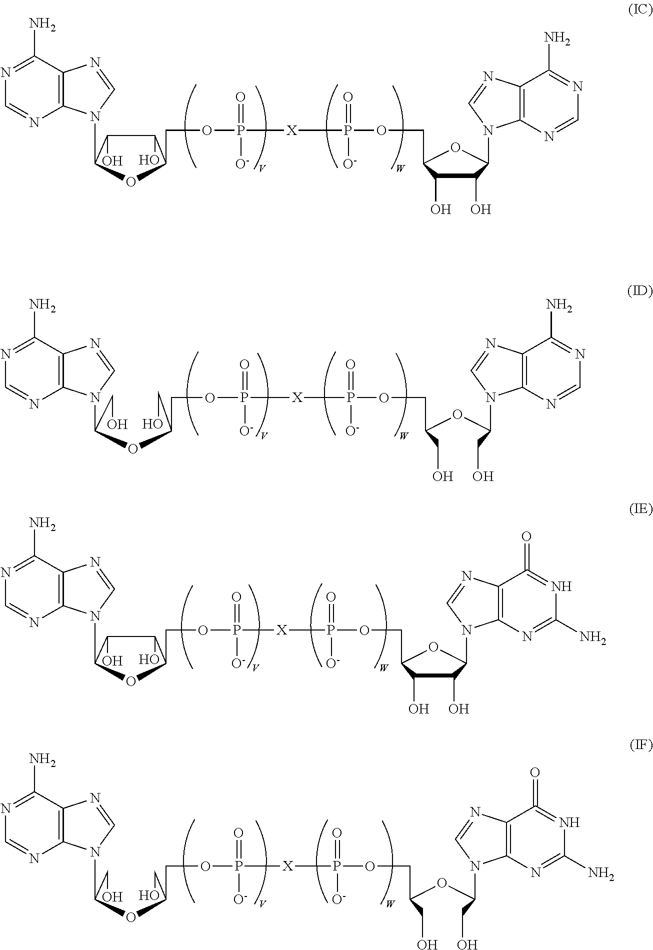

Preferably, the dinucleoside polyphosphate analogue is a compound of formula (IC) to (IF) wherein V plus W is 4 or 5. Thus, in a preferred aspect of the invention, the dinucleoside polyphosphate analogue is chosen among the group consisting of Ap₄A analogues, Ap₅A analogues, Ap₄G analogues and Ap₅G analogues.

In a preferred embodiment, V and W are the same. Thus in the above compounds of formula (I′) and (IA) to (IF), V and W are preferably each 2. In a further preferred embodiment, the dinucleoside polyphosphate analogue is symmetrical.

In a preferred aspect of the invention, the dinucleoside polyphosphate analogue is chosen among the group consisting of AppCH₂ppA, AppNHppA, A_{diol}ppCH₂ppA_{diol}, A_{diol}ppNHppA_{diol}, AppCH₂ppG, AppNHppG, A_{diol}ppCH₂ppG_{diol} and A_{diol}ppNHppG_{diol}:

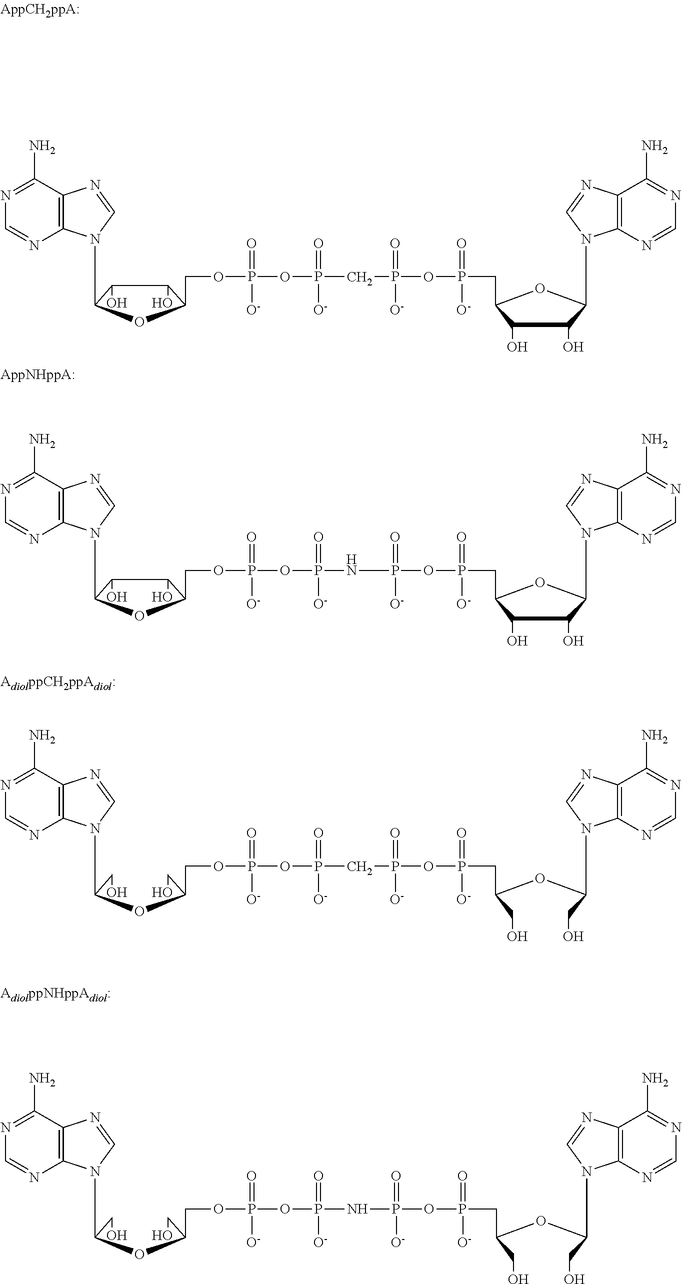

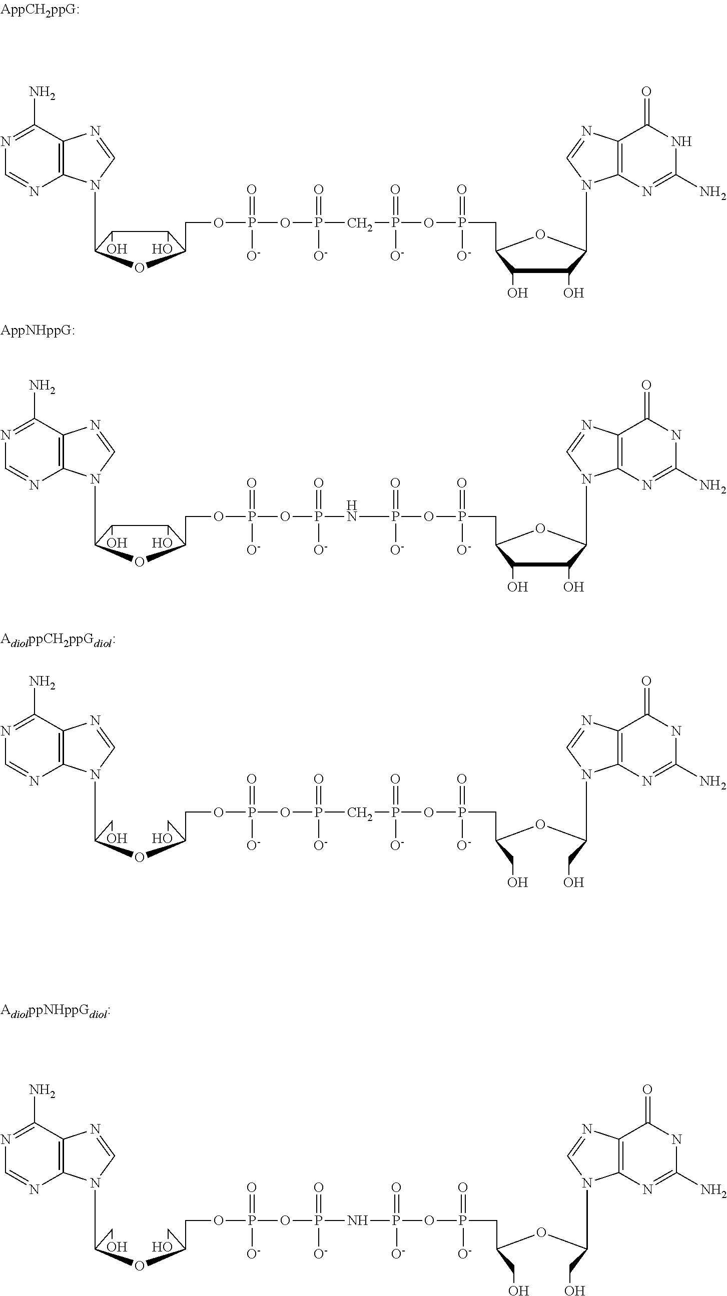

The dinucleoside polyphosphate analogues described herein have been found to potently inhibit or down-regulate P2X3 receptors via enhancement of desensitization and exert potent antinociceptive activities on an in vivo animal model of inflammatory pain (PCT/GB2013/051377). Thus these compounds have been found to be particulary effective in the treatment of pain, particulary moderate to chronic pain and/or back pain.

Dinucleoside polyphosphates of general formula (I) and their preparation are disclosed in WO 2006/082397.

### Salts and Anaesthetics

In one embodiment, the compound (for topical administration) according to the present invention comprises a pharmaceutically acceptable salt of a dinucleoside polyphosphate analogue. Preferably, the dinucleoside polyphosphate analogue is as described above.

The counter ion to the dinucleoside polyphosphate analogue may be any pharmaceutically acceptable counter ion. In a preferred embodiment, the counter ion is or comprises an anaesthetic (compound). For example, the composition may comprise a salt of a dinucleoside polyphosphate analogue as described herein with an anaesthetic compound selected from local anaesthetics (such as, but not limited to, an aminoester such as tetracaine, procaine, and benzocaine, or an aminoamide such as lidocaine, etidocaine and chinchocaine), and/or NSAIDS such as but not limited to the Coxib Etoricoxib.

Preferably, the composition comprises a salt of a dinucleoside polyphosphate analogue selected from AppCH₂ppA, AppNHppA, A_{diol}ppCH₂ppA_{diol}, A_{diol}ppNHppA_{diol}, A_{diol}ppNHppA_{diol}, AppCH₂ppG, AppNHppG, A_{diol}ppCH₂ppG_{diol} and A_{diol}ppNHppG_{diol} with an anaesthetic compound selected from local anaesthetics (such as but not limited to the aminoesters tetracaine, procaine, and benzocaine, or the aminoamides lidocaine, etidocaine and chinchocaine), and/or NSAIDS such as but not limited to the Coxib Etoricoxib.

Thus in one embodiment the present invention also relates to a compound that is a salt of a dinucleoside polyphosphate analogue and an anaesthetic compound, as described above, namely a compound comprising the analogue and an anaesthetic.

In one embodiment the present invention relates to a compound which comprises a dinucleoside polyphosphate analogue and an anaesthetic. This may be a salt of the dinucleoside polyphosphate analogue and anaesthetic compound, as described above, or the dinucleoside polyphosphate analogue and anaesthetic compound may be linked, for example via hydrogen bond(s). This may depend on the environment of the compound: for example it may be a salt in solution, but in the form of a hydrogen-bonded compound (e.g.) when formulated as a cream. The preferred dinucleoside polyphosphate analogues and anaesthetic compounds of the compound are as described above.

### Topical Administration

The pharmaceutical composition described herein is for topical administration. As used herein, topical administration refers to application to a body surface. Thus the compositions may be administered to the skin or an epithelial cell surface, such that the dinucleoside polyphosphate analogue (or a proportion thereof) can cross the relevant skin or epithelial cell barrier. The composition may have a local or systemic effect.

Suitably, the composition is in the form of a solution, cream, foam, gel, lotion or ointment. Preferably, the composition is a solution, cream or gel.

Preferably, the solution is an aqueous solution.

Topical cream delivery has been shown to be effective for delivery of nucleic acids, and would therefore be expected to be an advantageous route for delivery of the dinucleoside polyphosphate analogues of the present invention. For instance, GeneCream has been reported that penetrates the stratum corneum, and deposits nucleic acids such as siRNA in the epidermis, dermis, and to a lesser extent, subcutaneous tissue. When siRNA cream was topically applied to the skin of a collagen antibody-induced RA mouse model, the occurrence of severe, irreversible damage to bone and cartilage was reportedly reduced. Thus, the siRNA cream may represent a platform technology for delivery of siRNAs for treating various disorders including RA (Takanashi et al, 2009). An alternative is Imiquimod cream that was mixed with chitosan nanoparticles containing siRNA then applied to the skin of mice. The anti-inflammatory activity of transdermal siRNA was tested in OVA-sensitized mice by measuring airway hyperresponsiveness, eosinophilia, lung histopathology and pro-inflammatory cytokines. In a mouse asthma model, BALB/c mice treated with imiquimod cream containing siRNA-chitosan nanoparticles resulting in significantly reduced airway hyperresponsiveness, eosinophilia, lung histopathology and pro-inflammatory cytokines IL-4 and IL-5 in lung homogenates compared to controls. These results demonstrated that topical cream containing imiquimod and siRNA nanoparticles exerts an anti-inflammatory effect and may provide a new and simple therapy for asthma (Wang et al, 2008).

### Transdermal Delivery Devices

In another aspect, the present invention relates to devices for transdermal delivery, comprising a dinucleoside polyphosphate analogue or a pharmaceutically acceptable salt thereof. Such a physical delivery device can facilitate transport of compounds of interest into or across the skin barrier.

The device may be in the form of a patch containing the dinucleoside polyphosphate analogue and optionally a pharmaceutically acceptable excipient. The dinucleoside polyphosphate analogue may be dissolved, for example, in a gel and/or adhesive carrier on the patch. Suitable patch designs are well known, for example as described in U.S. Pat. No. 5,602,176, U.S. Pat. No. 6,316,023 or U.S. Pat. No. 6,335,031, which documents are fully incorporated by reference herein. A typical patch may comprise, in addition to the drug product in a matrix (e.g. an acrylic matrix): a backing film, and/or and layer comprising an adhesive (e.g. silicone) matrix, and/or a release liner (removed at time of use). Excipients within the formulation can include, for example, acrylic copolymer, poly(butylmethacrylate, methylmethacrylate), silicone adhesive applied to a flexible polymer backing film, silicone oil, and/or vitamin E.

Preferably, the device, preferably a patch, comprises a compound which is a salt of a dinucleoside polyphosphate analogue and an anaesthetic compound, or which comprises said analogue and an anaesthetic, wherein the dinucleoside polyphosphate analogue and an anaesthetic compound are preferably as described above.

Alternatively, the device (which may or may not be a patch) may comprise microneedles, for example in an array. Microneedles are typically no more than a micron in size: they may be able to penetrate the upper layer of the skin, for example without reaching nerves. The use of microneedles can thus facilitate transport of macromolecules across the skin barrier. Microneedles can be sharp and robust enough to easily penetrate the outer layer of skin. Due to their length can be such that they do not stimulate nerve cells deeper within the skin layers, the delivery of therapeutic agents can be pain-free. Furthermore, the use of microneedles can provide a slow release of the compounds to be delivered, since these are gradually released over time.

Preferably the microneedle-comprising device comprises a compound which is a salt of a dinucleoside polyphosphate analogue and an anaesthetic compound, or which comprises said analogue and an anaesthetic, wherein the dinucleoside polyphosphate analogue and an anaesthetic compound are preferably as described above.

In another embodiment, the device is an iontophoretic (transdermal) delivery device (or patch) comprising a pharmaceutically acceptable salt of a dinucleoside polyphosphate analogue. Such a device can make use of iontophoresis and/or electromotive drug administration (EMDA), to move or deliver the dinucleoside polyphosphate analogue (and any other compounds of interest) through or into the skin. Such a device enables efficient, non-invasive delivery of compounds of interest through or into the skin. It can thus cause the compound to flow diffusively (into or through the skin), for example as driven by an electric field. The device may be portable and/or attachable to the skin or body, e.g. similar to a Zecuity™ patch machine (used for migraine but can comprise compounds of the invention).

Preferred salts of the dinucleoside polyphosphate analogue for use in an iontophoretic transdermal delivery device are as described above.

The amount of the active agent (i.e. the dinucleoside polyphosphate analogue or pharmaceutically acceptable salt thereof, or compound which is a salt of a dinucleoside polyphosphate analogue and an anaesthetic compound, or which comprises said analogue and an anaesthetic) to be used in any of the devices as described above will vary depending on a number of factors, including the agent release characteristics of the pharmaceutical compositions, the active agent penetration rate observed in in vitro and in vivo tests, the potency of the active agent, the size of the skin contact area, the part of the body to which the unit is stuck, and the duration of action required. The skilled person would be able to determe determine the appropriate amount, for example by routine bioavailability tests.

Given the daily dose of active agent for oral administration, the choice of a suitable quantity of active agent to be incorporated in a device according to the invention will depend upon the pharmacokinetic properties of the active agent, including the first pass effect; the amount of active agent which can be absorbed through the skin from the matrix in question for a given area of application and in a given time; and the time for which the composition is to be applied. Thus, an active agent with a high first pass effect may require a relatively low quantity in the device for transdermal delivery when compared with the oral daily dose, since the first pass effect will be avoided. On the other hand, generally a maximum of only approximately 50% of the drug in the matrix is released through the skin in a 3 day period.

Suitable dosage amounts of the active agent of the present invention (i.e. the dinucleoside polyphosphate analogue or pharmaceutically acceptable salt thereof, or compound which is a salt of a dinucleoside polyphosphate analogue and an anaesthetic compound, or which comprises said analogue and an anaesthetic) are provided below. Equivalent dosages apply for any human subject, for example of weight 60 kg, 70 kg or 80 kg. The skilled person would be able to determine appropriate amounts for incorporation in a device for transdermal delivery based on this information and routine experimentation.

### Treatment

As described above, in one aspect the composition and device for transdermal delivery of the present invention are for use in treatment of the human or animal body by topical administration, i.e. to the skin or an epithelial cell surface of a human or animal subject. In view of the effects described above, the compositions or devices are preferably for use in the treatment of pain (or epilepsy, as a anticonvulsant and/or seizure suppressant).

Pain may be classified into different types. Nociceptive pain is mediated by pain receptors in response to injury, disease or inflammation. Neuropathic pain is a neurological disorder caused by damage to the pain transmission system from periphery to brain. Psychogenic pain is pain associated with actual mental disorder.

Pain may be chronic or acute, depending on its duration. Chronic pain can generally be described as pain that has lasted for a long time, for example beyond the expected period of healing. Typically, chronic pain is pain which lasts for 3 months or more. Pain which lasts for less than 30 days can be classed as acute pain, and pain of intermediate duration can be described as moderate or subacute pain.

The pain treated by the present invention may be associated with, for example, symptoms associated with one or more of inflammation (for example from cancer, arthritis or trauma), back pain (including sciatic back pain), trapped nerve, arthritic pain, cancer-related pain, dental pain, endometriosis, birthing-related pain (e.g. pre- and/or post-partum), post-surgical pain or trauma.

As described above, the dinucleoside polyphosphate analogues as described herein are particularly active against P2X3 receptors (especially homomeric P2X3 receptors), and in this respect PCT/GB2013/051377 is hereby incorporated, in its entirety, by reference. They can therefore be administered in low amounts compared with known agents for the treatment of pain.

Thus for the treatment (including prevention and/or reduction) of pain, the dinucleoside polyphosphate analogue is preferably administered in an amount of about 0.01 to 1000 nmol/kg, preferably from 0.1 to 500 nmol/kg, for example from 0.01 to 500 μg/kg, preferably from 0.1 to 250 μg/kg. In one embodiment, the dinucleoside polyphosphate analogue is preferably administered in an amount of from 0.01 to 10 μg/kg, preferably 0.05 to 5 μg/kg, more preferably from 0.1 to 2 μg/kg (i.e. a dose of 0.7 to 140 μg for a 70 kg human).

The dinucleoside polyphosphate analogue of the present invention is preferably administered in an amount of about 10 to 500 nmol/kg, preferably from 12 to 75 nmol/kg, more preferably from 25 to 50 nmol/kg. Thus for example the compound may be administered in an amount of from 6 to 100 μg/kg, preferably 10 to 75 μg/kg, more preferably from 12 to 50 μg/kg (i.e. a dose of 0.84 to 3.5 mg for a 70 kg human).

In one preferred embodiment of the present invention, the composition or device comprising a dinucleoside polyphosphate analogue are for use in treatment of moderate to chronic pain by administration to the skin or epithelial cell surface. The moderate to chronic pain may be mediated by nociceptive and/or neuropathic mechanisms. Preferably, the moderate to chronic pain may be nociceptive, for example, associated with at least one of the symptoms chosen among the group consisting of: inflammation (for example from cancer or arthritis), back pain, arthritic pain, cancer-related pain, dental pain, endometriosis and post-surgical pain. In particular, the moderate to chronic pain may be associated with inflammation, back pain, arthritis or cancer-related pain, particularly inflammation or cancer-related pain.

Thus, the present invention also relates to a composition or device comprising a dinucleoside polyphosphate analogue (as described herein) or a pharmaceutically acceptable salt thereof, for use in the treatment of moderate to chronic pain by administration to the skin or epithelial cell surface of a human or animal subject. In particular, the pain may be moderate to chronic neuropathic or moderate to chronic nociceptive pain, for example moderate to chronic nociceptive pain associated with at least one of the symptoms chosen among the group consisting of: inflammation (for example from cancer or arthritis), back pain, arthritic pain, cancer-related pain, dental pain, endometriosis and post-surgical pain. In particular, the moderate to chronic pain may be associated with inflammation, back pain, arthritis or cancer-related pain, particularly inflammation or cancer-related pain.

The present invention also relates to a method of treating moderate to chronic pain, comprising administering an effective amount of a composition comprising a dinucleoside polyphosphate analogue (as described herein) or a pharmaceutically acceptable salt thereof by administration to the skin or epithelial cell surface of a human or animal subject, and to use of a composition comprising a dinucleoside polyphosphate analogue (as described herein) or a pharmaceutically acceptable salt thereof, in the manufacture of a medicament for the treatment of moderate to chronic pain by administration to the skin or epithelial cell surface of a human or animal subject. In particular, the moderate to chronic pain is moderate to chronic neuropathic or moderate to chronic nociceptive pain, for example moderate to chronic nociceptive pain associated with at least one of the symptoms chosen among the group consisting of: inflammation (for example from cancer or arthritis), back pain, arthritic pain, cancer-related pain, dental pain, endometriosis and post-surgical pain. In particular, the moderate to chronic pain may be associated with inflammation, back pain, arthritis or cancer-related pain, particularly inflammation or cancer-related pain.

For the treatment of moderate to chronic pain, the dinucleoside polyphosphate analogue for use in the present invention is preferably administered in an amount of about 0.01 to 100 nmol/kg, preferably from 0.1 to 10 nmol/kg. Thus the compound may be administered in an amount of from 0.01 to 10 μg/kg, preferably 0.05 to 5 μg/kg, more preferably from 0.1 to 2 μg/kg.

Preferably, the dinucleoside polyphosphate analogue is one of the preferred analogues described above. In particular, the present invention relates to a composition comprising a dinucleoside polyphosphate analogue for use in the treatment of moderate to chronic pain by administration to the skin or epithelial cell surface of a human or animal subject, preferably wherein the dinucleoside polyphosphate analogue is chosen among the group consisting of: AppCH₂ppA, AppNHppA, A_{diol}ppCH₂ppA_{diol}, A_{diol}ppNHppA_{diol}, AppCH₂ppG, AppNHppG, A_{diol}ppCH₂ppG_{diol} and A_{diol}ppNHppG_{diol}.

For example, for a typical human of about 70 kg, the amount of the compound administered may be between about 1 and about 100 nmol, more preferably between about 10 and about 100 nmol, and even more preferably between about 10 and about 50 nmol.

In another embodiment, the composition or device comprising a dinucleoside polyphosphate analogue of the present invention are for use in the treatment of acute pain or subacute pain by administration to the skin or epithelial cell surface. Thus the present invention also relates to a method of treating acute pain or subacute pain, comprising administering an effective amount of a composition comprising a dinucleoside polyphosphate analogue (as described herein) or a pharmaceutically acceptable salt thereof by administration to the skin or epithelial cell surface, and to use of a composition comprising a dinucleoside polyphosphate analogue (as described herein) or a pharmaceutically acceptable salt thereof, in the manufacture of a medicament for the treatment of acute pain or subacute pain by administration to the skin or epithelial cell surface.

The acute pain or subacute pain may preferably be associated with post-surgical pain, dental pain, birthing-related pain, trauma or inflammation (for example resulting from trauma).

For the treatment of acute pain or subacute pain, the dinucleoside polyphosphate analogue is preferably administered in an amount of about 50 to 1000 nmol/kg, preferably from 50 to 500 nmol/kg, more preferably from 75 to 300 nmol/kg. Thus the compound may be administered in an amount of from about 10 to 500 μg/kg, preferably from 50 to 250 μg/kg.

Preferably, the dinucleoside polyphosphate analogue is one of the preferred analogues described above. In particular, the present invention relates to a composition comprising a dinucleoside polyphosphate analogue for use in the treatment of acute pain or subacute pain by administration to the skin or epithelial cell surface, preferably wherein the dinucleoside polyphosphate analogue is chosen among the group consisting of: AppCH₂ppA, AppNHppA, A_{diol}ppCH₂ppA_{diol}, A_{diol}ppNH_{PP}A_{diol}, AppCH₂ppG, AppNHppG, A_{diol}ppCH₂ppG_{diol} and A_{diol}ppNHppG_{diol}, preferably administered in the amounts described above.

### Nanoparticle(s)

In another aspect, the present invention relates to a pharmaceutical composition comprising a dinucleoside polyphosphate analogue or a pharmaceutically acceptable salt thereof combined with (e.g. linked to, inside, comprising, associated or formulated with or encapsulated within) a nanoparticle carrier, and a pharmaceutically acceptable excipient, or a (nano) particle comprising such an analogue (or salt). The dinucleoside polyphosphate analogue or a pharmaceutically acceptable salt thereof are preferably as described above.

The present invention may also relate to a pharmaceutical composition comprising a compound which comprises a dinucleoside polyphosphate analogue and an anaesthetic combined with (e.g. linked to, inside, comprising, associated or formulated with or encapsulated within) a nanoparticle carrier, and a pharmaceutically acceptable excipient, or a (nano) particle comprising such a compound. The dinucleoside polyphosphate analogue and an anaesthetic compound are preferably as described above.

Suitable exemplary nanoparticle carrier systems are lipid-based (or containing) nanoparticles, polymer-based (or containing) nanoparticles, inorganic nanoparticles and bioconjugates. The compound may be located in the core/centre or inside a lipid (bi)layer(s) which may be generally spherical. The particle may have multiple (e.g. concentric and/or spherical) layers as well, e.g. comprising lipids and/or polymers. The particle may be able to self-assemble. These are discussed in more detail below.

1.1 Lipid-Based, Synthetic ABC and ABCD Nanoparticles.

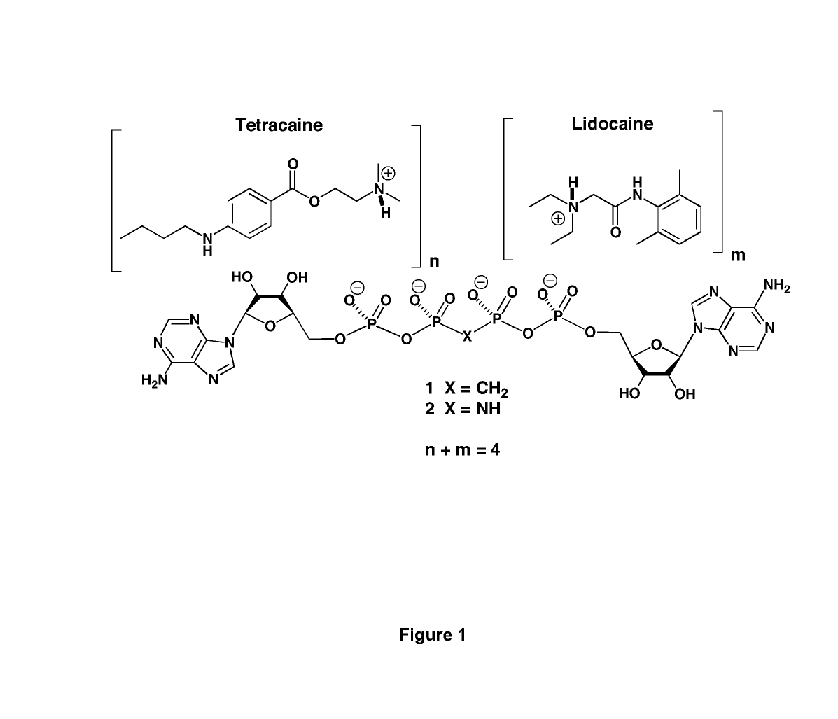

Safe, efficient synthetic nanoparticles for delivery of biopharmaceutical agents can be used. From a background in non-viral gene therapy¹⁻⁴, synthetic, self-assembly, ABC and ABCD nanoparticles have been configured specifically to mediate the functional delivery of active pharmaceutical ingredients (APIs) in vivo, such as small interfering RNA (siRNA) or plasmid DNA (pDNA)¹ (FIG. 1). Over the past few years, proprietary tool-kits of chemical components have been developed⁵⁻¹³, in order to set up the modular (“lego-model”) self-assembly of tailor-made, purpose designed ABC and ABCD nanoparticles (<100 nm in diameter, monodisperse). ABC nanoparticles set up for smart activation or triggerability (i.e., nanoparticles are stable in biological fluids but capable of mediating the controlled release of APIs in response to endogenous (or exogenously applied) changes in local conditions such as pH, t_{1/2} in highly interactive environments, redox state, local enzyme levels etc)¹⁴⁻¹⁸. For example, triggered ABC nanoparticles have been created and used to mediate the functional delivery of pDNA to lung, siRNA to liver and siRNA to tumour in vivo¹⁴⁻¹⁶. ABCD nanoparticles can be engineered for targeting (active D-components)^{12,13,19,20}. These will be upgraded with the potential for smart activation or triggerability as appropriate going forward.

Benefits of this LNP nanotechnology over other systems under development can be:
Hyperflexible, modular, scalable approach to nanoparticle assembly allowing for the formulation in principle of tailor-made nanoparticles of choice that can be targeted specifically to any desired site of interest.
Incorporation of triggerability into nanoparticles enabling these to be stable under normal circumstances, but triggered to disintegrate and release the payload (A-component) at a desired site of interest (pH, t_{1/2}, redox, enzyme, and thermal triggered release systems are the main technologies developed to date).
Flexible post-coupling chemistry that seeks to incorporate stealth/biocompatibility polymer (C-components) and optional targeting ligands (D-components) in a highly controlled and reproducible manner, giving rise to nanoparticles of very uniform composition and dimensions.

ABCD nanoparticles should be appropriate for clinical use going forward but the correct choices of targeting ligands relevant to diseases of interest will be essential. Data to date^{22,23} indicate that targeting ligands do not control nanoparticle biodistribution and API pharmacokinetics, but do promote improved pharmacodynamics. Current nanoparticle delivery systems require at least 100-fold improvement in pharmacodynamics for clinical use. The expectation is that this can be found with a judicious choice of nanoparticle platform and application of targeting ligands. This will be a major focus of our effort over the next few years.

1.2. Alternative LNP Systems.

LNP systems in general should be at or below 100 nm for successful functional delivery of nucleic acids in vivo in order to overcome various key biological barriers in vivo, for example the blood components, the reticuloendothelial system (RES) uptake, extracellular matrix components, and intracellular barriers. The major factors that impact the diameter and encapsulation efficiency of nucleic acid-containing LNPs include the lipid composition, nucleic acid to lipid ratio and formulation method. LNPs are often prepared using a dialysis method either from an aqueous-detergent or aqueous-organic solvent mixture. Alternative dehydration-rehydration followed by sonication and vortex mixing represents and alternative method. Irrespective, resulting LNPs have diameters about 100 nm and nucleic acid encapsulation efficiencies of >80%. LNPs typically require a PEG-surface coat to improve the particle pharmacokinetic behavior, a targeting ligand to facilitate target-cell recognition and in some case a bioresponsive lipid or pH-triggered polymer to enhance nucleic acid release and intracellular trafficking (Li & Szoka, 2007). A subset of LNPs that has barely been explored for nucleic acid delivery in vivo corresponds with microemulsion nanoparticles that are prepared traditionally through combination of micelle forming amphiphile with an oil-in-water mixture (Wu et al, 2001a; Wu et al, 2001b). This could be a fruitful area for future development for delivery of siRNA and smaller nucleotides to the skin.

### 2. Polymer-Based Nanoparticles (PNPs).

The functional delivery of nucleic acids such as siRNA may be assisted alternatively using polymer-based nanoparticles (PNPs). PNPs are formed by self-assembly of polycations with siRNA and can be used for site-specific delivery, cellular uptake and intracellular trafficking as a strategy to improve the therapeutic potential of siRNA. This is particularly true of systemic and mucosal routes of administration in vivo. There is a particular interest in the development of bioresponsive or stimuli-responsive systems that promote intracellular trafficking of siRNA (Howard & Kjems, 2007) (Kim & Kim, 2009) (De Rosa & La Rotonda, 2009; Fatal & Barratt, 2009).

2.1. Polyethylenimine (PEI)-Based Nanoparticles.

These have been widely studied as nucleic acid carriers, both, in vitro and in vivo. However, interest has recently developed in degradable polymeric systems. The advantage of degradable polymer is its low in-vivo cytotoxicity, which is a result of its easy elimination from the cells and body. Degradable polymer also enhances transfection of DNA or small interfering RNA (siRNA) for efficient gene expression or silencing, respectively (Jere et al, 2009b) (Jere et al, 2009a).

2.2. Alternative PNPs include nucleic acid/PEG-ε-caprolactone-malic acid (PEG-PCL/MA) nanoparticles. The intravenous injection of these PNPs has been used to control tumour growth based on siRNA delivery (Bouclier et al, 2008). Then there are the well-known poly-L-lysine based polymers nowadays enhanced with L-histidine residue inclusions. Proof of concept was demonstrated with poly-L-lysine partially substituted with L-histidine residues thereby promoting a dramatic increase in delivery efficacy of 3-4.5 orders of magnitude relative to poly-L-lysine controls. Moreover, several other histidine-rich polymers and peptides have been reported to be efficient carriers for the delivery of nucleic acids in vitro and in vivo. Such histidylated carriers are often only weakly cytotoxic in contrast to parent molecules (Midoux et al, 2009). Finally, there has been substantial recent interest in chitosan use, particularly to mediate siRNA delivery in vivo (Andersen et al, 2009).

2.3. Reduction-Sensitive Biodegradable Polymers.

These are seen as the preferred way forward where possible. The design rationale of reduction-sensitive polymers and conjugates usually involves incorporation of disulfide linkage(s) in the main chain, at the side chain, or in the cross-linker. Reduction-sensitive polymers are characterized by an excellent stability in the circulation and in extracellular fluids, whereas they are prone to rapid degradation under a reductive environment present in intracellular compartments such as the cytoplasm and the cell nucleus. This feature renders them distinct from their non-hydrolytically degradable counterparts and extremely intriguing for the controlled cytoplasmic delivery of a variety of bioactive molecules including nucleic acids. It is evident that reduction-sensitive biodegradable polymers and conjugates could be highly promising functional biomaterials (Meng et al, 2009).

2.4. Poly Lactide-Co-Glycolide (PLGA) Nanoparticles.

These have been known for a very long time as biodegradable nanocarrier systems. Nevertheless, applications to nucleic acid delivery have been limited until recent innovations in preparation methods (Braden et al, 2009) (Cun et al, 2010) (Khan et al, 2004). Alternatively cationic polymers such as PEI can be incorporated into PLGA particles by a spontaneous modified emulsification diffusion method. These hybrid nanoparticles are able to completely bind siRNA, provide protection for siRNA against nuclease degradation and mediate functional delivery of siRNA competitive with PEI-mediated delivery (Katas et al, 2009) (Patil & Panyam, 2009). In addition amine-modified-poly vinyl alcohol (PVA)-PLGA/siRNA nanoparticles have been reported. These PNPs achieved 80-90% knockdown of a luciferase reporter gene with only 5 pmol anti-luc siRNA, even after nebulization into murine lungs (Nguyen et al, 2008). In other innovations, PLGA nanoparticles can also be surface coated with chitosan for nucleic acid delivery using the emulsion solvent diffusion (ESD) method. The advantages of this method are a simple process under mild conditions without sonication. By coating the PLGA nanoparticles with chitosan, the nucleic acid loading efficiency was increased significantly (Tahara et al, 2008). In a similar way, cationic lipids (such as DOTAP, DOTMA, DC-Chol or CTAB) can also be present to promote the loading efficiency of nucleic acids (Takashima et al, 2007) (Tahara et al, 2008).

2.5. Nanogels.

These are swollen nanosized networks composed of hydrophilic or amphiphilic polymer chains. They are developed as carriers for the transport of drugs, and can be designed to spontaneously incorporate biologically active molecules through formation of salt bonds, hydrogen bonds, or hydrophobic interactions. Polyelectrolyte nanogels can readily incorporate oppositely charged low-molecular-mass drugs and biomacromolecules such as oligo- and polynucleotides (siRNA, DNA) as well as proteins. The guest molecules interact electrostatically with the ionic polymer chains of the gel and become bound within the finite nanogel. Multiple chemical functionalities can be employed in the nanogels to introduce imaging labels and to allow targeted drug delivery. The latter can be achieved, for example, with degradable or cleavable cross-links. Recent studies suggest that nanogels have a very promising future in biomedical applications (Kabanov & Vinogradov, 2009). Numbered within the nanogels are hydrogel scaffolds prepared from three different types of macroscopic, degradable biomaterials: calcium crosslinked alginate, photocrosslinked alginate, and collagen. These biopolymer hydrogels may entrap nucleic acids and are injectable, therefore, can be delivered in a minimally invasive manner, and they can serve as delivery vehicles for both nucleic acids and transplanted cell populations (Krebs et al, 2009).

### 3. Inorganic Nanoparticle Systems

3.1. Calcium Carbonate (CaCO₃) Nanoparticles.

These can be prepared e.g. with 58 nm average diameters. Both DNA and siRNA will complex with these nanoparticles and shown post administration to dramatically suppresses tumor lymphangiogenesis, tumor growth and regional lymph-node metastasis in subcutaneous xenografts (He et al, 2008) (He et al, 2009). Organic-inorganic hybrid-nanocarriers based, e.g. on the self-assembly of the block aniomer, poly(ethylene glycol)-block-poly(methacrylic acid), with calcium phosphate crystals that encapsulate nucleic acids (Kakizawa et al, 2006) can be used.

3.2. Calcium Phosphate (Ca₃(PO₄)₂) Nanoparticles.

Other reported inorganic hybrid carriers include single-shell calcium phosphate nanoparticles formed from rapid mixing of aqueous solutions of calcium nitrate and diammonium hydrogen phosphate. Multi-shell nanoparticle variants are possible, e.g. using added layers of calcium phosphate to protect nucleic acids from the intracellular degradation by endonucleases. The size of the these nanoparticles (according to dynamic light scattering and electron microscopy) was up to 100 nm (Kovtun et al, 2009). A lipid coated calcium phosphate (LCP) nanoparticle (NP) system can also be used, e.g. are developed for efficient delivery of nucleic acids such as small interfering RNA (siRNA) to a xenograft tumor model by intravenous administration. In an LCP-NP, a calcium phosphate core can condense nucleic acids covered by a surface lipid layer and supplementary PEG and targeting ligand layers. Ligand modified LCP-NPs can be used and can mediate efficient functional delivery of nucleic acids to a xenograft model (Li et al, 2010).

### 4. Bioconjugation

Active biological agents (such as siRNAs) and compounds can be chemically conjugated to a variety of bioactive molecules, lipids, and peptides to try to enhance their pharmacokinetic behavior, cellular uptake, target specificity, and safety. To efficiently deliver siRNAs to the target cells and tissues, many different siRNA bioconjugates have been synthesized and evaluated (Jeong et al, 2009). Results with bioconjugation generally suggest that nanoparticle mediated methodologies of delivery should be more widely applicable.

The compositions comprising nanoparticle carries are suitable for the same medical uses as those described above.

### Delivery

In one aspect, the compositions comprising a nanoparticle carrier may be administered orally, for example as tablets, troches, lozenges, aqueous or oily suspensions, dispersible powders or granules. The compositions may also be administered parenterally; for example subcutaneously, intravenously, intramuscularly, intrasternally, or by infusion techniques; or as suppositories. In particular, the compositions may be administered by subcutaneous injection.

The formulation of the composition will depend upon factors such as the nature of the exact agent, whether a pharmaceutical or veterinary use is intended, etc. An agent for use in the present invention may be formulated for simultaneous, separate or sequential use.

The compositions comprising a nanoparticle (carrier) may comprise the compound and calcium phosphate and/or Ca carbonate and are typically formulated for administration in the present invention with a pharmaceutically acceptable excipient (such as a carrier or diluents). The pharmaceutical carrier or diluent may be, for example, an isotonic solution. For example, solid oral forms may contain, together with the active compound, diluents, e.g. lactose, dextrose, saccharose, cellulose, corn or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tableting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Formulations for oral administration may be formulated as controlled release formulations, for example they may be formulated for controlled release in the large bowel.

Solutions for intravenous administration or infusion may contain as carrier, for example, sterile water or preferably they may be in the form of sterile, aqueous, isotonic saline solutions.

In another aspect, the compositions comprising a nanoparticle carrier may be administered topically. Thus, the compositions may be formulated for topical administration, for example as a solution, cream, foam, gel, lotion or ointment as described above.

Alternatively, the compositions comprising a nanoparticle carrier may be administered using a device for transdermal delivery, such as a patch or microneedle array, or other form of minimally invasive technique such as iontophoresis (Elsabahy M, Foldvari M: Needle-free gene delivery through the skin: an overview of recent strategies. Current Pharma Design, (2013) Mar. 12, manuscript in press).

The dose of the dinucleoside polyphosphate analogues may be determined according to various parameters, especially according to the substance used; the age, weight and condition of the patient to be treated; the route of administration; and the required regimen.

Again, a physician will be able to determine the required route of administration and dosage for any particular patient. A typical daily dose is from about 0.01 to 1000 μg per kg of body weight, according to the age, weight and conditions of the individual to be treated, the type and severity of the condition (e.g. of the pain) and the frequency and route of administration. Daily dosage levels may be, for example, from 0.01 to 500 μg/kg. In the treatment of moderate to chronic pain, suitable daily dosage levels may be from about 0.01 to 20 μg/kg, preferably from 0.05 to 15 μg/kg, preferably from 0.1 to 10 μg/kg. In the treatment of acute pain or subacute pain, suitable daily dosage levels may be from about 10 to 1000 μg/kg, preferably from 50 to 500 μg/kg.

The dinucleoside polyphosphate analogues as described herein may be administered alone or in combination. They may also be administered in combination with another pharmacologically active agent, such as another agent for the treatment of pain, for example an opioid, non-opioid or NSAID. For example, the dinucleoside polyphosphate analogues for use according to the present invention may be combined with an opioid such as oxycodone (for example OxyContin®; controlled-release oxycodone HCl; Purdue Pharma L.P.). The combination of agents may be may be formulated for simultaneous, separate or sequential use.

All publications and patent applications mentioned in this specification are indicative of the level of those skilled in the art to which this invention pertains. All publications and patent applications are herein incorporated by reference to the same extent as if each individual publication or patent application was specifically and individually to be incorporated by reference.

Although the foregoing invention has been described in some detail by way of illustration and example for purposes of understanding, it will be clear to those skilled in the art that certain changes and modifications may be practiced within the scope of the appended claims.

The following Examples illustrate the invention.

### EXAMPLES

### Example 1

AppCH₂ppA and AppNHppA are both tetraacidic and so may form pharmaceutically acceptable salts in combination with monobasic aminoester local anesthetics such as tetracaine, and/or with monobasic aminoamide local anesthetics such as lidocaine (FIG. 1). These salts may be administered by direct injection, by patch or in combination with minimially invasive techniques such as iontophoresis or microneedles (Elsabahy M, Foldvari M: Needle-free gene delivery through the skin: an overview of recent strategies. Current Pharma Design, (2013) Mar. 12, manuscript in press).

### Example 2

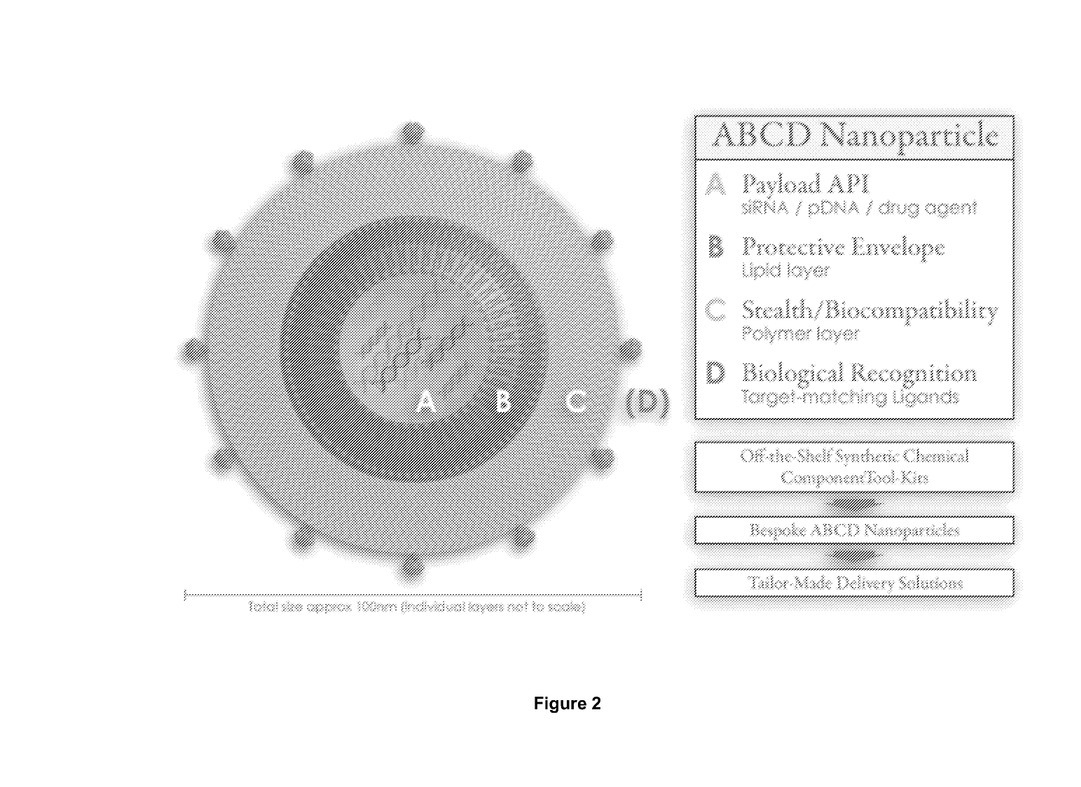

AppCH₂ppA and AppNHppA are both tetraacidic and may be combined (in the form of salts as above or as free acid) in ABC/ABCD lipid-based nanoparticle systems (LNPs) for transdermal delivery. Appropriate formlulations can be derived with reference to some of the latest literature on formulation of small interfering RNA (siRNA) and other RNA interference (RNAi) effectors or DNA into ABC/ABCD LNPs (Miller A D (2013) Delivery of RNAi therapeutics: work in progress. Expert Rev. Med. Devices 10: 781-811) (FIG. 2). These LNP formulations may then be delivered transdermally by direct injection, by patch or in combination with minimially invasive techniques such as iontophoresis or microneedles (Elsabahy M, Foldvari M: Needle-free gene delivery through the skin: an overview of recent strategies. Current Pharma Design, (2013) Mar. 12, manuscript in press; Rodriguez-Cruz I M, et al. Polymeric nanospheres as strategy to increase the amount of triclosan retained in the skin: passive diffusion vs. iontophoresis, J Microencap (2013) 30, 72).

### Example 3

A patch of area 10 cm² is prepared, by preparing a composition comprising:
(a) 0.2-2 mg of a compound as described in Example 1, wherein said compound constitutes 20% of the composition by weight,
(b) 30% by weight of a hydrophilic polymer, e.g. Eudragit E 100™,
(c) 44% by weight of a non swellable acrylate polymer, e.g. Durotack 2802416™, and
(d) 6% by weight of a plasticizer, e.g. Brij 97™

These components are added to acetone or ethanol or another appropriate volatile organic solvent and mixed to give a viscous mass. The mass is spread on top of an aluminised polyester foil (thickness 23 microns) using a conventional apparatus, to produce a film of thickness 0.2 mm when wet. The film is allowed to dry at room temperature over 4 to 6 hours. The aluminium foil is then cut up into patches about 10 sq cm in area.

FIG. 1 Illustration of pharmaceutically acceptable salts of AppCH₂ppA and AppNHppA with tetracaine and lidocaine.

FIG. 2 In ABCD LNPs, active pharmaceutical ingredients (APIs, e.g., dinucleoside polyphosphates) (A) are condensed within functional concentric layers of chemical components designed for delivery into cells and intracellular trafficking (B components—lipids), biological stability (C stealth/biocompatibility components—typically Polyethylene Glycol [PEG]) and biological targeting to target cells (D biological targeting ligand components).

### REFERENCES

### ABC/ABCD Nanoparticle References

1. Kostarelos, K.; Miller, A. D. Chem. Soc. Rev. 2005, 34, 970; 2. Miller, A. D. Curr Med. Chem. 2003,10, 1195; 3. Miller, A. D. ChemBioChem 2004, 5, 53; 4. Miller, A. D. Methods Mol. Med. 2004, 90, 107; 5. Tagawa, T. et al. Gene Ther. 2002, 9, 564; 6. Oliver, M. et al. Tetrahedron Lett. 2004, 45, 3105; 7. Fletcher, S. et al. Org. Biomol. Chem. 2006, 4, 196; 8. Fletcher, S. et al. J. Med. Chem. 2006, 49, 349; 9. Spagnou, S. et al. Biochemistry 2004, 43, 13348; 10. Fletcher, S. et al. ChemBioChem, 2008, 9, 455; 11. Mével, M. et al. J. Control. Rel. 2010, 143, 222; 12. Andreu, A. et al. ChemBioChem, 2008, 9, 219; 13. Wang, M. et al. Bioconjugate Chem. 2009, 20, 32; 14. Carmona, S. et al. Mol. Pharmaceutics 2009, 6, 706; 15. Aissaoui, A. et al. J. Control. Rel., 2011, 154, 275; 16. Kenny, G. D. et al. J. Control Rel. 2011, 149, 111; 17. Drake, C. R. et al. Mol. Pharmaceutics 2010, 7, 2040; 18. In addition please note Kolli, S. et al. Bioconjugate Chem. 2013, 24, 314; Yingyuad, P. et al., Bioconjugate Chem. 2013, 24, Drake, C. R. et al. 2012, J. Control. Rel. 2013, 171, 81; 19. Chen, J. et al. J. RNAi Gene Silencing, 2011, 7, 449; 20. In addition please note the following: Wang, M. et al. J. Drug Target. 2013, 21, 684; 21. Oliver, M. et al. Org. Biomol. Chem. 2006, 4, 3489; 22. Kamaly, N. et al. Bioconjugate Chem. 2008, 19, 118; 23. Kamaly, N. et al. Bioconjugate Chem. 2009, 20, 648; 24. Kamaly, N. et al. Org. Biomol. Chem. 2010, 8, 201.

### General References

Andersen M O, Howard K A, Kjems J (2009) RNAi using a chitosan/siRNA nanoparticle system: in vitro and in vivo applications. Methods Mol Biol 555: 77-86
Barry B W (2001) Novel mechanisms and devices to enable successful transdermal drug delivery. European journal of pharmaceutical sciences: official journal of the European Federation for Pharmaceutical Sciences 14: 101-114
Bouclier C, Moine L, Hillaireau H, Marsaud V, Connault E, Opolon P, Couvreur P, Fatal E, Renoir J M (2008) Physicochemical characteristics and preliminary in vivo biological evaluation of nanocapsules loaded with siRNA targeting estrogen receptor alpha. Biomacromolecules 9: 2881-2890
Braden A R, Kafka M T, Cunningham L, Jones H, Vishwanatha J K (2009) Polymeric nanoparticles for sustained down-regulation of annexin A2 inhibit prostate tumor growth. J Nanosci Nanotechnol 9: 2856-2865
Cun D, Foged C, Yang M, Frokjaer S, Nielsen H M (2010) Preparation and characterization of poly(DL-lactide-co-glycolide) nanoparticles for siRNA delivery. Int J Pharm 390: 70-75
De Rosa G, La Rotonda M I (2009) Nano and microtechnologies for the delivery of oligonucleotides with gene silencing properties. Molecules 14: 2801-2823
Escobar-Chavez J J, Bonilla-Martinez D, Villegas-Gonzalez M A, Molina-Trinidad E, Casas-Alancaster N, Revilla-Vazquez A L (2011) Microneedles: a valuable physical enhancer to increase transdermal drug delivery. Journal of clinical pharmacology 51: 964-977
Fatal E, Barratt G (2009) Nanotechnologies and controlled release systems for the delivery of antisense oligonucleotides and small interfering RNA. Br J Pharmacol 157: 179-194
Garland M J, Migalska K, Mahmood T M, Singh T R, Woolfson A D, Donnelly R F (2011) Microneedle arrays as medical devices for enhanced transdermal drug delivery. Expert review of medical devices 8: 459-482
Gasperlin M, Gosenca M (2011) Main approaches for delivering antioxidant vitamins through the skin to prevent skin ageing. Expert Opin Drug Deliv 8: 905-919
Geusens B, Strobbe T, Bracke S, Dynoodt P, Sanders N, Van Gele M, Lambert J (2011) Lipid-mediated gene delivery to the skin. European journal of pharmaceutical sciences: official journal of the European Federation for Pharmaceutical Sciences 43: 199-211
Gonzalez-Rodriguez M L, Rabasco A M (2011) Charged liposomes as carriers to enhance the permeation through the skin. Expert Opin Drug Deliv 8: 857-871
Gupta P N, Vyas S P (2011) Colloidal carrier systems for transcutaneous immunization. Curr Drug Targets 12: 579-597
He X W, Liu T, Chen Y X, Cheng D J, Li X R, Xiao Y, Feng Y L (2008) Calcium carbonate nanoparticle delivering vascular endothelial growth factor-C siRNA effectively inhibits lymphangiogenesis and growth of gastric cancer in vivo. Cancer Gene Ther 15: 193-202
He X W, Liu T, Xiao Y, Feng Y L, Cheng D J, Tingting G, Zhang L, Zhang Y, Chen Y X (2009) Vascular endothelial growth factor-C siRNA delivered via calcium carbonate nanoparticle effectively inhibits lymphangiogenesis and growth of colorectal cancer in vivo. Cancer Biother Radiopharm 24: 249-259
Howard K A, Kjems J (2007) Polycation-based nanoparticle delivery for improved RNA interference therapeutics. Expert Opin Biol Ther 7: 1811-1822
Jeong J H, Mok H, Oh Y K, Park T G (2009) siRNA conjugate delivery systems. Bioconjug Chem 20: 5-14
Jere D, Arote R, Jiang H L, Kim Y K, Cho M H, Cho C S (2009a) Bioreducible polymers for efficient gene and siRNA delivery. Biomed Mater 4: 25020
Jere D, Jiang H L, Arote R, Kim Y K, Choi Y J, Cho M H, Akaike T, Cho C S (2009b) Degradable polyethylenimines as DNA and small interfering RNA carriers. Expert Opin Drug Deliv 6: 827-834
Kabanov A V, Vinogradov S V (2009) Nanogels as pharmaceutical carriers: finite networks of infinite capabilities. Angew Chem Int Ed Engl 48: 5418-5429
Kakizawa Y, Furukawa S, Ishii A, Kataoka K (2006) Organic-inorganic hybrid-nanocarrier of siRNA constructing through the self-assembly of calcium phosphate and PEG-based block aniomer. J Control Release 111: 368-370
Katas H, Cevher E, Alpar H O (2009) Preparation of polyethyleneimine incorporated poly(D,L-lactide-co-glycolide) nanoparticles by spontaneous emulsion diffusion method for small interfering RNA delivery. Int J Pharm 369: 144-154
Khan A, Benboubetra M, Sayyed P Z, Ng K W, Fox S, Beck G, Benter I F, Akhtar S (2004) Sustained polymeric delivery of gene silencing antisense ODNs, siRNA, DNAzymes and ribozymes: in vitro and in vivo studies. J Drug Target 12: 393-404
Kim W J, Kim S W (2009) Efficient siRNA delivery with non-viral polymeric vehicles. Pharm Res 26: 657-666
Kovtun A, Heumann R, Epple M (2009) Calcium phosphate nanoparticles for the transfection of cells. Biomed Mater Eng 19: 241-247
Krebs M D, Jeon O, Alsberg E (2009) Localized and sustained delivery of silencing RNA from macroscopic biopolymer hydrogels. J Am Chem Soc 131: 9204-9206
Li J, Chen Y C, Tseng Y C, Mozumdar S, Huang L (2010) Biodegradable calcium phosphate nanoparticle with lipid coating for systemic siRNA delivery. J Control Release 142: 416-421
Li W, Szoka F C, Jr. (2007) Lipid-based nanoparticles for nucleic acid delivery. Pharm Res 24: 438-449
Meng F, Hennink W E, Zhong Z (2009) Reduction-sensitive polymers and bioconjugates for biomedical applications. Biomaterials 30: 2180-2198
Midoux P, Pichon C, Yaouanc J J, Jaffres P A (2009) Chemical vectors for gene delivery: a current review on polymers, peptides and lipids containing histidine or imidazole as nucleic acids carriers. Br J Pharmacol 157: 166-178
Neubert R H (2011) Potentials of new nanocarriers for dermal and transdermal drug delivery. Eur J Pharm Biopharm 77: 1-2
Nguyen J, Steele T W, Merkel O, Reul R, Kissel T (2008) Fast degrading polyesters as siRNA nano-carriers for pulmonary gene therapy. J Control Release 132: 243-251
Patil Y, Panyam J (2009) Polymeric nanoparticles for siRNA delivery and gene silencing. Int J Pharm 367: 195-203
Polat B E, Hart D, Langer R, Blankschtein D (2011) Ultrasound-mediated transdermal drug delivery: mechanisms, scope, and emerging trends. J Control Release 152: 330-348
Prow T W, Grice J E, Lin L L, Faye R, Butler M, Becker W, Wurm E M, Yoong C, Robertson T A, Soyer H P, Roberts M S (2011) Nanoparticles and microparticles for skin drug delivery. Adv Drug Deliv Rev 63: 470-491
Romero E L, Morilla M J (2011) Topical and mucosal liposomes for vaccine delivery. Wiley interdisciplinary reviews Nanomedicine and nanobiotechnology 3: 356-375
Russell-Jones G, Himes R (2011) Water-in-oil microemulsions for effective transdermal delivery of proteins. Expert Opin Drug Deliv 8: 537-546
Sachdeva V, Banga A K (2011) Microneedles and their applications. Recent Pat Drug Deliv Formul 5: 95-132
Shah U U, Roberts M, Orlu Gul M, Tuleu C, Beresford M W (2011) Needle-free and microneedle drug delivery in children: a case for disease-modifying antirheumatic drugs (DMARDs). Int J Pharm 416: 1-11
Singh T R, Dunne N J, Cunningham E, Donnelly R F (2011) Review of patents on microneedle applicators. Recent Pat Drug Deliv Formul 5: 11-23
Swain S, Beg S, Singh A, Patro Ch N, Rao M E (2011) Advanced techniques for penetration enhancement in transdermal drug delivery system. Curr Drug Deliv 8: 456-473
Tahara K, Sakai T, Yamamoto H, Takeuchi H, Kawashima Y (2008) Establishing chitosan coated PLGA nanosphere platform loaded with wide variety of nucleic acid by complexation with cationic compound for gene delivery. Int J Pharm 354: 210-216
Takanashi M, Oikawa K, Sudo K, Tanaka M, Fujita K, Ishikawa A, Nakae S, Kaspar R L, Matsuzaki M, Kudo M, Kuroda M (2009) Therapeutic silencing of an endogenous gene by siRNA cream in an arthritis model mouse. Gene Ther 16: 982-989
Takashima Y, Saito R, Nakajima A, Oda M, Kimura A, Kanazawa T, Okada H (2007) Spray-drying preparation of microparticles containing cationic PLGA nanospheres as gene carriers for avoiding aggregation of nanospheres. Int J Pharm 343: 262-269
Van Gele M, Geusens B, Brochez L, Speeckaert R, Lambert J (2011) Three-dimensional skin models as tools for transdermal drug delivery: challenges and limitations. Expert Opin Drug Deliv 8: 705-720
Wang X, Xu W, Mohapatra S, Kong X, Li X, Lockey R F, Mohapatra S S (2008) Prevention of airway inflammation with topical cream containing imiquimod and small interfering RNA for natriuretic peptide receptor. Genet Vaccines Ther 6: 7
Wohlrab J, Kreft B, Tamke B (2011) Skin tolerability of transdermal patches. Expert Opin Drug Deliv 8: 939-948
Wu H, Ramachandran C, Bielinska A U, Kingzett K, Sun R, Weiner N D, Roessler B J (2001a) Topical transfection using plasmid DNA in a water-in-oil nanoemulsion. Int J Pharm 221: 23-34
Wu H, Ramachandran C, Weiner N D, Roessler B J (2001b) Topical transport of hydrophilic compounds using water-in-oil nanoemulsions. Int J Pharm 220: 63-75

1. A pharmaceutical composition for topical administration, comprising a dinucleoside polyphosphate analogue or a pharmaceutically acceptable salt thereof and a pharmaceutically acceptable excipient.

2. Composition according to claim 1, wherein said dinucleotide polyphosphate analogue is a compound of formula (I): 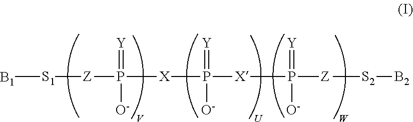
or a pharmaceutically acceptable salt thereof, wherein X, X′
and Z are independently selected from 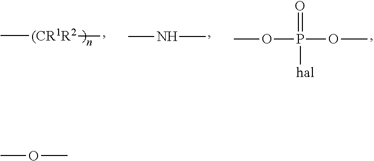
wherein R¹ and R² are independently selected from hydrogen, halogen, hydroxyl, cyano or an unsubstituted group selected from C₁₋₃ haloalkyl, C₁₋₃ alkyl, C₁₋₄ aminoalkyl and C₁₋₄ hydroxyalkyl, and n is selected from 1, 2, 3, 4, 5 and 6;
each Y is independently selected from ═S and =0;
B₁ and B₂ are independently selected from a 5- to 7-membered carbon-nitrogen heteroaryl group which may be unfused or fused to a further 5- to 7-membered carbon-nitrogen heteroaryl group
S₁ and S₂ are independently selected from a bond, C₁₋₆ alkylene, C₂₋₆ alkenylene, C₂₋₆ alkynylene and a moiety of formula (II): 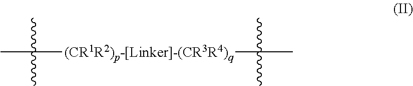
wherein
R¹, R², R³ and R⁴ independently represent hydrogen, halogen, hydroxyl, cyano or an unsubstituted group selected from C₁₋₃ haloalkyl, C₁₋₃ alkyl, C₁₋₄ aminoalkyl and C₁₋₄ hydroxyalkyl;
p and q independently represent 0, 1, 2 or 3, preferably 0, 1 or 2; and [Linker] represents:
(i) —O—, —S—, —C═O— or —NH—;
(ii) C₁₋₄ alkylene, C₂₋₄ alkenylene or C₂₋₄ alkynylene, which may
optionally contain or terminate in an ether (—O—), thioether (—S—), carbonyl (—C═O—) or amino (—NH—) link, and which are optionally substituted with one or more groups selected from hydrogen, hydroxyl, halogen, cyano, —NR⁵R⁶ or an unsubstituted group selected from C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₂₋₄ aminoalkyl, C₁₋₄ hydroxyalkyl, C₁₋₄ acyl and C₁₋₄ alkyl-NR⁵R⁶ groups, wherein R⁵ and R⁶ are the same or different and represent hydrogen or unsubstituted C₁₋₂ alkyl; or (iii) a 5 to 7 membered heterocyclyl, carbocyclyl or aryl group, which may be optionally substituted with one or more groups selected from hydrogen, hydroxyl, halogen, cyano, —NR⁵R⁶ or an unsubstituted group selected from C₁₋₄ alkyl, C₂₋₄ alkenyl, C₁₋₄ alkoxy, C₂₋₄ alkenyloxy, C₁₋₄ haloalkyl, C₂₋₄ haloalkenyl, C₁₋₄ aminoalkyl, C₁₋₄ hydroxyalkyl, C₁₋₄ acyl and C₁₋₄ alkyl-NR⁵R⁶ groups, wherein R⁵ and R⁶ are the same or different and represent hydrogen or unsubstituted C₂₋₄ alkyl;
V is selected from 0, 1, 2, 3, 4 and 5;
U is selected from 0, 1, 2, 3, 4 and 5;
W is selected from 0, 1, 2, 3, 4 and 5; and
V plus U plus W is an integer from 2 to 7.

3-65. (canceled)
